(19) United States
(12) Patent Application Publication
(10) Pub. No.: US 2024/0180123 A1
(43) Pub. Date: Jun. 6, 2024

(54) Method and Product for the Identification of Diseased Animals

(71) Applicant: Ioannis Sideris, Athens (GR)

(72) Inventor: Ioannis Sideris, Athens (GR)

(21) Appl. No.: 18/526,027

(22) Filed: Dec. 1, 2023

(30) Foreign Application Priority Data
Dec. 6, 2022 (GR) 20220101009

(51) Int. Cl. A01K 11/00; A01K 29/00

(52) U.S. Cl. CPC A01K 11/00; A01K 29/005

(57) ABSTRACT

A product for identifying a diseased animal within a group of animals includes a plurality of capsules, each of the plurality of capsules comprising a film encapsulating a plurality of beads; wherein the film and each of the plurality of beads of the capsule bear a unique distinguishing feature that differentiates the capsule from the remaining capsules in the plurality of capsules.

### CROSS-REFERENCE TO RELATED APPLICATIONS

The instant application claims priority to Greek Patent Application No. 20220101009, filed Dec. 6, 2022, which is incorporated herein in its entirety by reference.

### FIELD OF THE DISCLOSURE

The present relates to diagnostic tools for identifying diseased animals and, more particularly, to diagnostic tools for use in diagnosing disease in farms and other animal production environments and populations, and in domestic animals.

### BACKGROUND OF THE INVENTION

Effectively managing the health and welfare of a herd of animals is a very important challenge for the sustainability of livestock production. The growing demand for safe and high-quality food of animal origin, the globalization of trade and the increasing cross-border spread of infectious diseases, require early diagnosis and control of biosecurity threats. Rapid, simple, simple, cheap, and reliable on-farm diagnosis of diseases in farm animals is an essential tool for the development of effective disease control methods. In order to achieve this, it is first necessary to identify the diseased animal and separate it from the herd before more members of the herd become infected. Consequently, detection and testing tools are strategically important tools based on the principles of animal husbandry. Identification of the diseased animal is also imperative for the early diagnosis of outbreaks of endemic or epidemic diseases, which may otherwise have serious socio-economic consequences.

Tools for identification and diagnosis are a critical link between two key strategies for good health, prevention and treatment. Veterinary diagnostic tools are critical to animal health, identifying health problems early and supporting faster diagnosis and planning for treatment. In breeding units and farms with a large population of productive animals, in herds with a large number of animals, in households with numerous pets, etc., where many animals live together in the same area, it is easy to transmit infectious diseases from one animal to another, especially when there are no obvious symptoms at the beginning of the disease, so that if it is not immediately identified which animal is sick within the group, there is a risk of spreading and transmitting the disease to the whole herd or even to all pets in the household. In addition, some of the diseases affecting livestock are zoonotic diseases, which are a public health concern, making it imperative to identify the specific animal in the herd that is diseased in a timely and immediate manner. The technical problem that arises is that the breeder or pet owner becomes aware from certain signs (e.g., vomiting, feces, etc.) that one of the animals is diseased, but it is difficult to identify that animal in a timely and accurate manner.

The current state of the art DE1010242, describes methods and compositions related to a system for releasing active ingredients into the stomach or gastrointestinal tract of animals, allowing easy and unambiguous marking of the vomit of the animal to identify those that are diseased among healthy animals residing in the same area. The active substances or constituents referred to in document DE1010242 play the role of marker, differentiating the vomit of the sick animal from that of the healthy ones, and are insoluble substances, chemical compounds or not, which cannot be absorbed by the gastrointestinal tract of the animal, monomeric or polymeric, and which are excreted by the diseased animal unchanged or modified, colorless or colored, which thereby mark and/or color the contents of the gastrointestinal tract and consequently mark and/or color the vomit. This identifies the affected animal.

Entry of the active tracer/marker components into the gastrointestinal tract of an animal shall is described to be by oral administration in an appropriate pharmaco-technical form when the findings will be detected in the vomit. As demonstrated above, with the current technique it is difficult to identify the diseased animal when it lives in a large population, both because the color palette is limited and because the colors are altered within the animal's body. According to the current state of the art, because of the limited number of colors, which must be perfectly distinct from each other, the owner or breeder or stock breeder must divide his pets or herds into several smaller ones. For example, in the case of four perfectly distinct different colors, such as blue, yellow, green, and black, each herd will have a maximum of five animals (four animals, one for each different color and an additional animal without color in the tracking marker). There is also the possibility of misinterpretation of the colors due to mixing with the rest of the stomach contents. This makes it very difficult for the owner, breeder, or stockman, both in the management of everyday life such as housing, feeding, etc. and in the quick identification of the diseased animal and its removal from the rest to avoid the spread of the disease.

In addition, only monogastric animals such as pets and companion animals and pigs (swine) vomit. In contrast, multi-gastric animals, or production animals, such as cows, lambs, cattle, etc., ruminalize and do not vomit. In this case, gastrointestinal or other disorders or diseases or illnesses are detected at an early stage, and only through the feces, especially diarrhea.

On the other hand, when non-degradable materials are used, they remain only in the soil and pollute the environment and the ecosystem. Most modern synthetic materials do not have bacteria that can dissolve them, so they remain intact over time, polluting the environment.

### BRIEF SUMMARY OF THE INVENTION

In one general aspect, the present disclosure describes a method and product for rapidly and reliably identifying a diseased animal in groups with a large number (population) of productive or companion animals without the assistance of a veterinarian. The method and product in accordance with the disclosure does not constitute a diagnostic method or product, but rather a product and method for identifying a particular animal in a group of animals based on the animal's secretions.

In one embodiment, the product according to the disclosure is in the form of a suppository for multi-gastric production animals such as cattle, sheep, goats, and the like, and a suppository and/or capsule for pets (domestic animals), e.g., cats, dogs etc. or monogastric production animals such as pigs or wild boars (swine). The size and form of the product according to the invention varies according to the animal type, size, age, and the like.

In one particular embodiment, the suppository is formed as a capsule having a film coating onto which a specific number is printed or engraved. The capsule contains discernible beads bearing the same number printed or engraved on the capsule such that the beads are discernible when presented loose and not within the original capsule. The goal is to identify the beads when secreted by the animal and correlate them to the capsule consumed or otherwise deposited into the animal. In this sense, other bead identifiers may also be used. For example, the capsules may have a monochromatic or multi-chromatic film that is indicative of the color or color combinations of beads that are contained therein. For example, a capsule may be formed by blue and green film enclosing blue and green beads.

The number or color arrangement of film and beads is correlated to a particular animal that ingested or otherwise incorporated or used the particular bead or beads. Thus, when the diseased animal vomits or has diarrhea, it will expel the numbered or colored beads together with the contents of its stomach. By observing the beads, the number written on them, or the color(s) of the beads, can be identified and correlated to the animal to which it corresponds, and which is diseased, can be identified.

In addition, in order not to burden the environment with additional plastic pollution, the numbered and/or colored beads contained in the formulation are made of biodegradable materials, non-absorbable by the body. The film encapsulating the beads is dissolvable and absorbable by the animal so that the beads can be released to facilitate their identification later. In a healthy animal, the beads will be excreted by the animal in the normal course of its digestive process.

Advantages of the embodiments in accordance with the disclosure are that it solves the aforementioned technical problem and achieves fast and accurate identification of the diseased animal (one or more) in farms and/or herds with a large population quickly and easily avoiding further separation into smaller sub-herds or sub-groups and achieving early detection and timely treatment of the problem. This is because the number and/or color combinations of the beads that are enclosed in the capsules is theoretically limitless. It also achieves the isolation of sick animals and the prevention of disease transmission to healthy animals in contact with the sick animals and to humans. The method is particularly useful both for the timely treatment of the sick animal and for the protection of healthy animals, as most of the time the identification of sick animals is very difficult and costly and time consuming due to non-apparent symptoms.

A further advantage is that the embodiments in accordance with the disclosure avoid any case of error due to misinterpretation of regurgitated or excreted colored liquid and/or any alterations of the same (e.g., yellow with green), as with the current technique there is a possibility that the color of the vomit may be misinterpreted due to the mixing of the stomach contents with the pigment. In contrast, the embodiments in accordance with the present disclosure do not present the risk of misinterpretation of the finding, as the number written on the numbered beads is 100% legible, and/or the color of the beads is unchanged and unalterable, and in both cases the number or color of the beads cannot be misinterpreted.

Suitable materials for manufacturing the beads described in the context of the present disclosure are materials that, after use, gradually decompose thanks to the intervention of living beings (bacteria) such as fungi and other microorganisms that exist in nature. Thanks to this, they do not stick to the soil or any medium and do not contaminate. The decomposition process starts with the bacteria that extract the enzymes and favor the conversion of the original product into simpler elements. Finally, all the small particles in the soil are gradually absorbed.

### BRIEF DESCRIPTION OF THE SEVERAL VIEWS OF THE DRAWING(S)

FIG. 1 is a schematic view of illustrates a film-coated pill/suppository enclosing numbered beads of biodegradable materials in a numbered film in accordance with the disclosure.

FIGS. 2A and 2B are two film-coated pills/suppositories, enclosing numbered beads of biodegradable materials in a numbered film, intended for use on farms with more than one herd with a large number of animals in accordance with the disclosure.

### DETAILED DESCRIPTION OF THE INVENTION

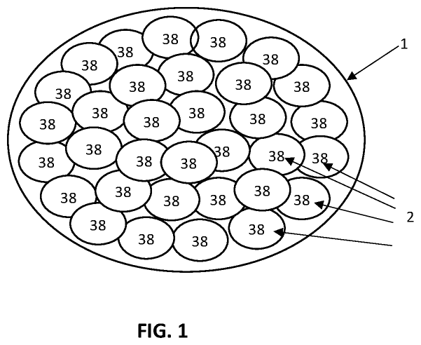

As illustrated in FIG. 1, an embodiment of a product in accordance with the disclosure can be manufactured in the form of a suppository for multi-gastric production animals, e.g., cattle and sheep and goats, and in the form of a suppository and/or capsule for small domestic animals (pets), e.g., cats, dogs, etc. or monogastric production animals such as pigs or wild boars (swine). The product is manufactured with a film (1) of material dispersible in the animal's body and enclosing within it, as a core, beads (2) of non-absorbable but biodegradable material. Both the film and the beads it encloses shall be numbered or otherwise appropriately labelled for identification, for example, colored. The beads of a pill/suppository and the film shall all bear the same number or other appropriate distinguishing marking. Alternatively, the film and beads can be made with matching colors or color combinations. Each animal shall have a different pill, bearing a different number or other appropriate distinguishing markings, both on the film as well as the beads. For conducting the diagnosis, the pill(s) are provided to the animals for ingestion and/or supposition.

During use, a first animal may be provided by a pill where the film and beads are marked with the number 1, the second animal uses the pill where the film and beads are marked with the number 2, and so on. In the example illustrated in FIG. 1, the pill illustrated has a film and beads engraved with the number 38 and is intended to be used on a farm with a population of up to 60 animals. At the same time, since each production animal has its own identity and is marked with a unique number or code that is applied to the animal immediately after its birth, (e.g., as an earring), it becomes easier to identify and link it to the number of the pill/suppository to be used on that animal. Thus, in the example above, the number 38 will correspond exclusively to a particular animal. When the animal becomes ill and vomits or has diarrhea, it will expel the numbered beads along with the stomach contents. By observing the beads, the number written on them can be identified and the animal to which the beads correspond, and which is the animal that is sick can be identified.

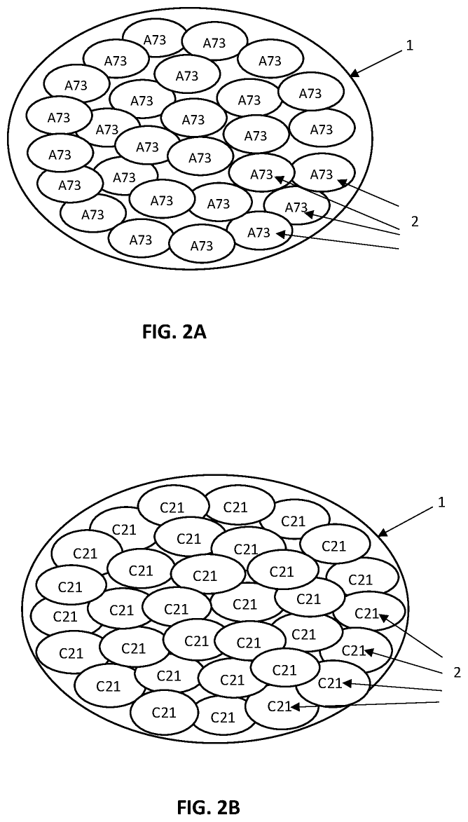

In a second example where a farm has more than one herd, a letter can be printed on the beads and on the film, so that a distinction can be made between the herds (e.g., A73 and C21). Thus, in the example illustrated in FIG. 2, the product is used on a farm with a population of up to 320 animals, divided into 4 herds of up to 80 animals each. In this case,
in herd A, film-coated pills are used in all animals in which the film and the beads (2) in the film (1) are numbered A1 to A80,
in herd B, film-coated pills with and beads (2) numbered B1 to B80,
in herd C, film-coated pills and beads (2) numbered C1 to C80, and
in herd D, film-coated pills and beads (2) numbered D1 to D80.

Before or at the time of administration, the unique matching of each pill with the identity of the animal to which it is administered shall be recorded. This recording, in combination with the numbering of the beads, leads to the immediate identification of the diseased animal, even within large herds, with no margin for error.

The size and form of the product according to the invention may vary depending on the animal, its size and its age. Furthermore, any case of error due to misinterpretation of color and/or any alterations (e.g. yellow with green) thereof is also avoided, as the number or other appropriate distinctive marking indicated on the pellet is 100% legible and unalterable and cannot be misinterpreted under any circumstances.

The film consists of absorbent material. The numbered beads contained in the formulation are produced from biodegradable materials. Depending on the size of the production animal, the formulation targeted at a cow may be the size of a tennis ball, or if it is a lamb, respectively the size of a ping-pong ball. Within the formulation, the numbered beads shall be sized appropriately to accommodate a sufficient number of beads for easy identification and quick identification of the number they carry (e.g., 30 to 100 numbered pellets per formulation/pill).

The numbering or marking of the film (1) shall be done by printing in ecologically absorbent ink, or by engraving, or by any other appropriate method. The numbering or marking of the beads (2) shall be done by any suitable method, such as printing, engraving, or any other equivalent method. The number or other appropriate distinctive marking on each bead (2) may be either printed in indelible ecological ink so that it does not disappear when in contact with gastric or other liquids, or engraved on the bead for the same purpose, or displayed on the bead by any other appropriate method.

The embodiments in accordance with the disclosure facilitate and enable monitoring of an unlimited number of animals of one or more herds of a farm or production unit or breeding farm, due to the possibility of printing or engraving any number, or combination of number and letters, and/or colors, or other suitable distinctive marking.

In the manufacture of the product according to the invention, the product is in the shape/form of a sphere or cylinder or cone or other shape and/or form and is surrounded by a thin yet durable film of absorbent material.

Indicatively, in the case of a polymeric material, the polymer forms a flexible film and may have some or all of the following characteristics:
1. solubility in the chosen solvent (e.g., water),
2. solubility in aqueous media appropriate to the purpose of the coating (e.g., pH-dependent solubility in the case of enteric coating),
3. ability to produce a product of good appearance,
4. stability in the presence of heat, light, moisture, air and in contact with core materials
5. compatibility with the usual adjuvants added to the coating solution,
6. stability to fracture and satisfactory barrier properties to moisture, light, odor, and
7. case of printing on high-speed machines, to name a few.

Indicative examples of polymers that can be used to make the film of the invention include, but are not limited to: Hydroxypropyl methyl cellulose (HPMC); Methyl hydroxyethyl cellulose (HEMC); Ethyl cellulose (EC) for slow-release tablets; Hydroxypropyl cellulose (HPC); Povidone (povidone, PVP); Polyethylene glycol (PEG); Acrylic polymers (polymethacrylates), and others.

In one embodiment, the numbered beads or beads with other suitable distinctive marking are made of biodegradable materials. Biodegradable materials are considered to be all those materials that decompose due to the intervention of fungi and other microorganisms that exist in nature. When a substance is attacked by bacteria, the decomposition process begins, which extracts enzymes to convert the original product into simpler elements. The final stage consists of the gradual absorption of the particles by the soil.

Embodiments of a product according to the disclosure invention take advantage of scientific progress, use ecologically sustainable and biodegradable materials that can replace those that are now obsolete and harmful. In this way, the accumulation of harmful materials that occurs daily in the environment can be significantly reduced.

The beads of the product according to the invention can be manufactured using the following biodegradable materials, for example:
1. Starch and rye plastics—Biodegradable plastics from corn or wheat starch are currently produced on an industrial scale, for example for the manufacture of garbage bags. The degradation of these plastics can take 6 to 24 months, underground or in water, depending on the speed at which the starch is incorporated.
2. Biodegradable synthetic and natural plastics—In this group, there are certain types of synthetic polymers that can be broken down naturally or by the addition of substances that can accelerate their degradation. These plastics include oxygen biodegradable plastics and poly(e-caprolactone) (PCL). Oxidative biodegradable plastics are synthetic plastics in which chemical additives that promote oxidation are added to the formulation to initiate or accelerate the oxidative degradation process to produce biodegradable products. PCL is a biodegradable and biocompatible thermoplastic polyester used in medical applications.

The embodiments in accordance with the disclosure are applicable to farms with many and large herds as well as to small herds or domestic animals (pets), to monogastric animals such as companion animals as well as pigs (swine), but also to multi-gastric animals or otherwise production animals such as cows, lambs, cattle, etc.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of identifying a diseased animal within a group of animals, comprising:
administering, to each animal in the group of animals, a capsule selected from a plurality of capsules, the capsule comprising a film encapsulating a plurality of beads, wherein the film and each of the plurality of beads of the capsule bear a unique distinguishing feature that differentiates the capsule from the remaining capsules in the plurality of capsules;
correlating the capsule to a particular animal within the group of animals;
repeating the administering of different capsules from the plurality of capsules to different animals from the group of animals, and repeating the correlating of the different capsules with the different animals; and
when the diseased animal excretes the beads from its respective capsule as vomit or diarrhea, identifying the diseased animal based on the distinguishing features of the excreted beads.

2. The method of claim 1, further comprising sub-dividing a large group of animals into smaller groups of animals, wherein the distinguishing feature further includes an indication of one of the smaller groups of animals.

3. The method of claim 1, wherein administering the capsule is accomplished by providing the capsule to the animal for ingestion by mouth.

4. The method of claim 1, wherein administering the capsule is accomplished by inserting the capsule into the animal as a suppository.

5. The method of claim 1, wherein the film is ingestible by the animal.

6. The method of claim 1, wherein the plurality of beads is made from a biodegradable material.

7. The method of claim 1, wherein the distinguishing feature is an alphanumeric indication formed by printing or engraving processes.

8. A product for identifying a diseased animal within a group of animals, comprising:
a plurality of capsules, each of the plurality of capsules comprising a film encapsulating a plurality of beads;
wherein the film and each of the plurality of beads of the capsule bear a unique distinguishing feature that differentiates the capsule from the remaining capsules in the plurality of capsules.

9. The product of claim 8, wherein, during use, the capsule is correlatable to a particular animal within the group of animals, and wherein the capsule is administered to the particular animal.

10. The product of claim 9, wherein the capsule is configured for administration to the particular animal for ingestion by mouth.

11. The product of claim 9, wherein the capsule is configured for administration to the particular animal as a suppository.

12. The product of claim 8, wherein the film is ingestible by the animal.

13. The product of claim 8, wherein the plurality of beads is made from a biodegradable material.

14. The product of claim 8, wherein the distinguishing feature is an alphanumeric indication formed by printing or engraving processes.
